(19) United States
(12) Patent Application Publication
(10) Pub. No.: US 2009/0254026 A1
(43) Pub. Date: Oct. 8, 2009

(54) Syringe Device Comprising a Motor Adapted for Filling an Injection Chamber

(75) Inventor: Torben Strom Hansen, Bagsvaerd (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/299,524

(22) PCT Filed: May 3, 2007
(86) PCT No.: PCT/EP2007/054288

§ 371 Date: May 4, 2009

Related U.S. Application Data
(60) Provisional application No. 60/818,747, filed on Jul. 6, 2006.

(30) Foreign Application Priority Data
May 4, 2006 (EP) 06009240.0

(51) Int. Cl. A61M 5/30

(52) U.S. Cl. 604/68

(57) ABSTRACT

The present application relates to a syringe device comprising an injector chamber and a medicament container each comprising a piston. In particular the present application relates to a device comprising motor arranged to move an injector piston and/or a reservoir piston so as to transfer a medicament in the medicament container to the injector chamber. Furthermore, the present application relates to a syringe device comprising a motor arranged to strain the spring for assisting the ejection action. Moreover, the present application relates to the use of a motor to strain a spring of a syringe device.

### FIELD OF THE INVENTION

The present invention relates to a syringe device comprising an injector chamber and a medicament container each comprising a piston. In particular the present invention relates to a device comprising motor arranged to move an injector piston and/or a reservoir piston so as to transfer a medicament in the medicament container to the injector chamber. Furthermore, the present invention relates to a syringe device comprising a motor arranged to strain the spring for assisting the ejection action. Moreover, the present invention relates to the use of a motor to strain a spring of a syringe device.

### BACKGROUND OF THE INVENTION

Jet injection devices are filled by transferring a medicament from a medicament container to an injection chamber of the jet injection device via a fluid connection. In some embodiments of jet injection devices, the cross sectional area of such a fluid connection is small in order to prevent that the medicament during ejection, is forced back into the medicament container. However, one disadvantage of such narrow fluid connections is that transfer of the medicament from the medicament container to the injection chamber requires a relatively large force which makes it difficult for a user with poor dexterity to transfer the medicament.

Accordingly, it is an object of a preferred embodiment of the present invention to provide a jet injection device easing the medicament transfer.

Background art may be seen in U.S. Pat. No. 5,080,648, U.S. 2003/040715, U.S. 2004/015126, U.S. Pat. No. 3,507,276, WO 2006/078400, WO 2004/060143 and U.S. Pat. No. 6,210,420.

Moreover, in order to assist a patient in administering a medicament with a syringe device, devices having injection assisting means have been developed. Such devices may comprise a spring which is strained by the user prior to injection, whereby energy is accumulated, which may be released during injection in order to assist the user in injecting a medicament.

One example of such a device is known from U.S. 2005/0119608 A1 which discloses a needlefree injection device, including a plunger and a spring operatively coupled with the plunger. The spring is configured to be compressed during arming of the injection device, and decompressed during discharge to forcibly advance the plunger within a fluid chamber. The injection device further includes an actuator operable to cause the spring to be compressed during arming of the injection device.

However, even straining of a spring may be difficult or even impossible for a person with poor dexterity. Accordingly, it is an object of an embodiment of the present invention to improve the abovementioned devices such that a user is exempted from straining a spring of the syringe device. Moreover, it is an object of an embodiment of the present invention to provide a syringe device wherein the spring is strained correctly each time such that it is not strained too much or too little.

### BRIEF DESCRIPTION OF THE INVENTION

In a FIRST aspect the present invention relates to a syringe device comprising:
an injector body for accommodation of a medicament to be ejected;
an injector piston movable within the injector body such that when moved in a distal direction a medicament contained in an injector chamber defined by the injector body and the injector piston is expelled; and
a medicament container comprising a reservoir for accommodation of a medicament and a reservoir piston movable within the reservoir;

wherein the reservoir is fluidly connected to the injector chamber by means of a fluid connection; and

wherein a motor is coupled to the injector piston and/or the reservoir piston such that activation of the motor causes at least one of said pistons to move whereby a medicament contained in the reservoir flows into the injector chamber.

By providing a motor arranged to move at least one of the pistons the user may be assisted in filling the injection chamber with the medicament contained in the medicament container. This is advantageous for users with poor dexterity.

In the context of the present invention the term “syringe device” shall be understood as a device for administration of a substance such as a medicament, into the skin, subcutaneous tissue, muscle, blood vessels or body cavities, of a living being, with or without a cannula/needle.

In one embodiment the injector chamber is used for high pressure delivery of a medicament wherein the medicament is injected needleless at a pressure above 100 bar.

The injector body may define an outlet through which the medicament is expelled. The outlet may be substantially cylindrical and have a cross-sectional area below 1 mm2, such as below 0.1 mm², below 0.05 mm², below 0.03 mm². The length of the outlet may be below 10 mm, such as below 5 mm, such as below 2 mm, such as below 1 mm.

The fluid connection between the injector chamber and the reservoir may comprise a unidirectional valve preventing fluid contained in the injector chamber from flowing into the reservoir.

The reservoir piston rod may be coupled to the motor via a first coupling such that upon rotation of a drive shaft of the motor, the reservoir piston rod forces the reservoir piston in a distal direction whereby a medicament contained in the reservoir is forced through the fluid connection and into the injector chamber. In one embodiment the first coupling comprises a gear mechanism such as a threaded engagement between the reservoir piston rod and a drive shaft of the motor.

Moreover, the motor may be coupled to the injector piston via a second coupling comprising the gear mechanism such that upon activation of the motor, the injector piston is moved in a proximal direction whereby the volume of the injector chamber is increased and a medicament contained in the reservoir is forced into the injector chamber by suction.

In one embodiment the motor is coupled to both the first and the second coupling whereby operation of the motor causes the reservoir piston to be forced in the distal direction while at the same time the injector piston is moved in the proximal direction.

In one embodiment, the fluid connection forms a conduit in the injector piston and defines an outlet on a distal surface of the injector piston. Alternatively, or as a supplement, the fluid connection forms an outlet on an inner surface of the injector body.

In order to maintain substantially the same pressure in the injector chamber, the fluid connection and the reservoir during setting of a dose—wherein a medicament contained in the reservoir is forced into the injector chamber—the first and the second couplings may be dimensioned such that when the injector piston is moved in the proximal direction, the reservoir piston is moved in the distal direction at a speed ensuring that the increase in volume of the injector chamber approximately corresponds to the decrease in volume of the reservoir.

In one embodiment the syringe device comprises a spring adapted to be strained so as to store energy, the spring cooperating with the injector piston such when the spring is released from its strained state, the injector piston is forced in the distal direction. Moreover in said embodiment, the motor may be arranged to strain the spring so as to accumulate energy.

By providing a motor arranged to strain the spring, a user is exempted from straining the spring, which often requires good dexterity. Moreover, the device according to the present invention prevents incorrect i.e. too much or too little, straining of the spring, which may lead to incorrect injection pressure.

In order to provide a compact configuration of the device, the spring may encircle the reservoir such that a centre axis of the spring coincides with a centre axis defined by the reservoir. Moreover, the motor may define a cavity for accommodation of the reservoir piston rod.

In one embodiment, the motor is coupled to the spring via a gear mechanism. Accordingly, by choosing a sufficiently high gear ratio the motor may be adapted to provide a relatively small torque such as below 700 Nmm, such as below 500 Nmm, such as below 300 Nmm, or such as below 100 Nmm.

It will be appreciated that by choosing a high gear ratio and small torque, the time needed to strain the spring is relatively large. However, in connection with most diseases the time lapsing between two injections is counted in hours rather than seconds, and, thus, sufficient time is available to strain the spring even with high gear ratios. Thus, in one embodiment the gear ratio is chosen such that the straining time i.e. the time needed for the motor to strain the spring for the next injection, is counted in minutes such as 2 minutes, such as 5 minutes, such as 10 minutes, such as 15 minutes or even 30 minutes. Moreover, the gear mechanism may have a gear ratio of at least 4, such as at least 8, such as at least 12, such as at least 20.

In the context of the present invention the term “gear ratio” shall be understood as the relationship between the number of teeth on two gear wheels that are meshed directly of via one or more further gear wheels.

The spring may be arranged to be strained rotationally and/or translationally so as to store energy.

In order to release the energy accumulated by the spring, the device may comprise a release mechanism adapted to be changed between a retaining position wherein it retains the spring in its strained state, and a releasing position wherein the spring is allowed to be unstrained. The release mechanism may be operable from an outer surface of the device.

In one embodiment the release mechanism cooperates with a trigger provided on a distal surface of the device such that when the device is pressed towards the skin of a user, the release mechanism is changed from its retaining position to the releasing position, whereby the medicament contained in the injector chamber is expelled.

In a SECOND aspect, the present invention relates to the use of a motor to strain a spring of a syringe device according to the first aspect of the invention. The device used in the second aspect of the invention may comprise any combination of features and/or elements of the first aspect of the invention.

### DETAILED DESCRIPTION OF THE INVENTION

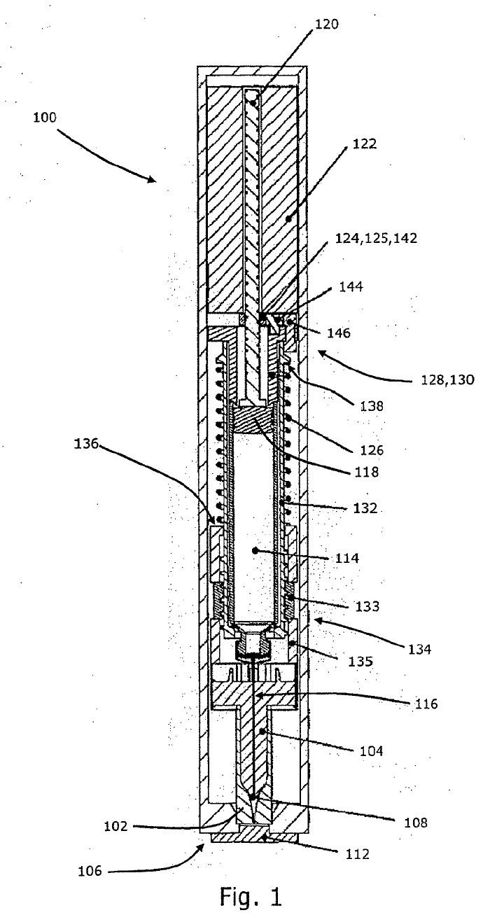

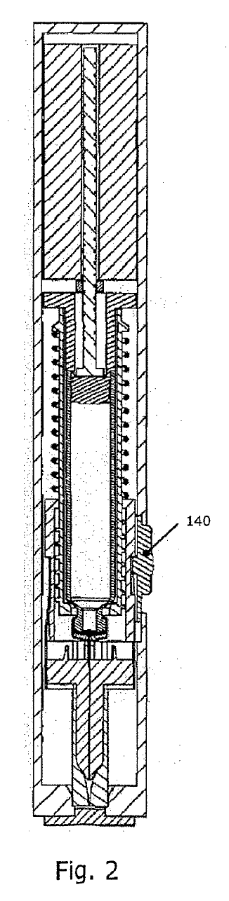

FIGS. 1 and 2 disclose a syringe device according to the present invention, and

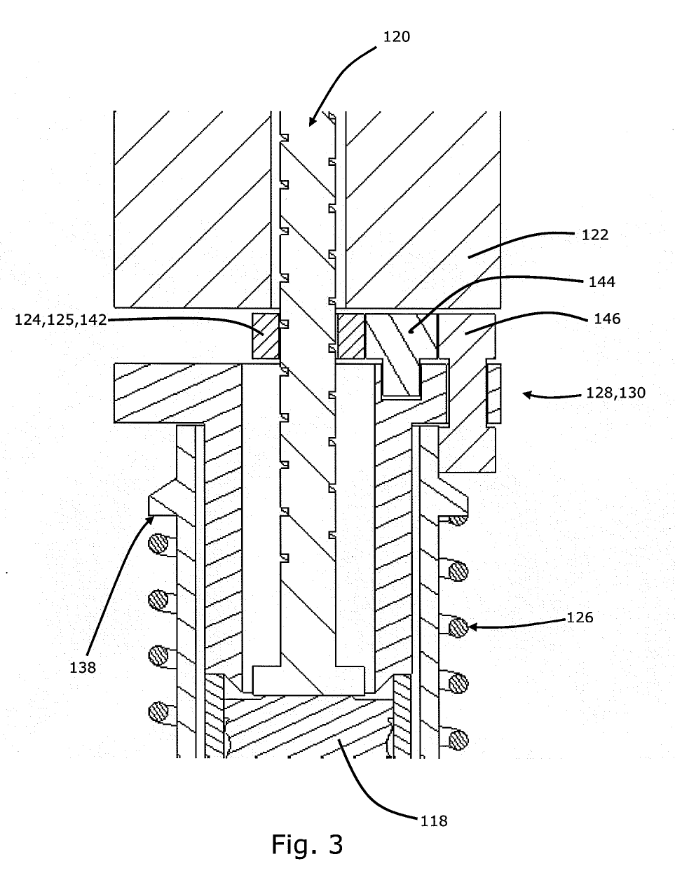

FIG. 3 discloses a motor coupled to a spring via a gear mechanism.

FIGS. 1 and 2 discloses a syringe device 100 comprising an injector body 102 and an injector piston 104 which is movable within the injector body 102 such that when moved towards a distal end 106 of the device 100 (i.e. downwards in the drawing), a medicament contained in an injector chamber 108 defined by the injector body 102 and the injector piston 104, is expelled through an outlet 110. It will be appreciated that in order to inject the medicament into the tissue of a living being, a protecting cover 112 of the device 100 must be removed. The injector chamber 108 is fluidly connected to a reservoir 114 by means of a fluid connection 116 which is defined in the injector piston 104. A reservoir piston 118 is movable within the reservoir 114 such that when the reservoir piston 118 is moved towards the distal end 106, a medicament contained in the reservoir 114 is forced through the fluid connection 116 and into the injector chamber 108. The reservoir piston 118 may be moved towards the distal end by means of a reservoir piston rod 120 which abut a proximal end of the reservoir piston 118. The reservoir piston rod 120 is movable by means of a motor 122 which is coupled to the reservoir piston rod 120 by means of a first coupling 124 comprising a piston rod gear 125. Moreover, the motor 122 is coupled to a spring 126 by means of a second coupling 128 comprising a gear mechanism 130 the details of which are disclosed in FIG. 3.

When the motor is operated, rotation of a drive shaft of the motor (not illustrated) is transferred to a spindle 132 via the gear mechanism 130. Due to threaded engagement between the spindle 132 and a ram assembly 134, the ram is moved in the proximal direction. As the ram assembly 134 and the injector piston 104 are coupled to each other, the injector piston is also moved in the proximal direction, whereby the volume of the injector chamber 108 is increased. Due to the increase in volume, the pressure in the injector chamber 108 decreases whereby a medicament contained in the reservoir 114 is sucked into the injector chamber 108. Additionally, the reservoir piston is moved in the distal direction due to the first coupling interconnecting the reservoir piston rod 120 and the motor 122. When the reservoir piston 118 is moved in the distal direction, the volume of the reservoir decreases whereby a medicament contained in the reservoir is forced through the fluid connection 116 and into the injector chamber 108. Accordingly, the medicament is transferred from the reservoir 114 to the injector chamber 108 by means of suction and overpressure due to movement of the injector piston 104 and the reservoir piston 118, respectively.

It will be appreciated that preferably the first and the second coupling should be designed such that the when the motor is operated, the decrease in the volume of the reservoir corresponds to the increase in the volume of the injector chamber, whereby the sum of the volumes defined by the reservoir, the injector chamber and the fluid connection remains substantially constant. Additionally, it will be appreciated that any difference in the inner diameter of the reservoir and the inner diameter of the injector body may be compensated for by choosing an appropriate first and second gear mechanisms which are adapted to move the reservoir piston and the injector piston at different speeds so as to increase/decrease the aforementioned volumes at substantially the same rate.

The ram assembly comprises a threaded lock 133 and non-threaded ran element 135. The ram element 135 is connected to the injector piston 104 and defines a distal abutment surface 136 which the spring abuts. Moreover, the spring 126 abut a proximal abutment surface 138 defined by the spindle 132. Accordingly, as the ram assembly 134 is moved in the proximal direction the spring 126 is strained translationally, and energy is accumulated by the spring.

The energy accumulated in the strained spring 126 is released by activating a release button 140 (see FIG. 2) whereby the threaded lock 133 is brought out of engagement with the outer threaded surface of the spindle 132. As a result the strained spring forces the ram assembly and the injector piston 104 in the distal direction, and the medicament contained in the injector chamber is expelled through the outlet 110.

As described above, the motor 122 is coupled to a spring 126 by means of a second coupling 128 comprising a gear mechanism 130. The gear mechanism 130 which is discloses in detail in FIG. 3, comprises first, second and third gear wheels 142, 144, 146. Due to threaded engagement between the first and the second gear wheel 142, 144 and between the second gear wheel and the third gear wheel 144, 146 and between the third gear wheel and the spindle 132, operation of the motor 122 causes the spindle to rotate, as the motor is coupled to the first gear wheel 142.

1. A syringe device comprising:
an injector body for accommodation of a medicament to be ejected;
an injector piston movable within the injector body such that when moved in a distal direction a medicament contained in an injector chamber defined by the injector body and the injector piston is expelled; and
a medicament container comprising a reservoir for accommodation of a medicament and a reservoir piston movable within the reservoir;
wherein the reservoir is fluidly connected to the injector chamber by means of a fluid connection; and
wherein a motor is coupled to the injector piston and/or the reservoir piston such that activation of the motor causes at least one of said pistons to move whereby a medicament contained in the reservoir flows into the injector chamber.

2. A syringe device according to claim 1, comprising a spring adapted to be strained so as to store energy, the spring cooperating with the injector piston such when the spring is released from its strained state, the injector piston is forced in the distal direction; and wherein the motor is arranged to strain the spring so as to accumulate energy.

3. A syringe device according to claim 1, further comprising a reservoir piston rod coupled to the motor via a first coupling such that upon rotation of a drive shaft of the motor, the reservoir piston rod forces the reservoir piston in a distal direction whereby a medicament contained in the reservoir is forced through the fluid connection and into the injector chamber.

4. A syringe device according to claim 1, wherein the motor is coupled to the injector piston via a second coupling comprising the gear mechanism such that upon activation of the motor the injector piston is moved in a proximal direction whereby the volume of the injector chamber is increased and a medicament contained in the reservoir is forced into the injector chamber by suction.

5. A syringe device according to claim 1, wherein the fluid connection forms a conduit in the injector piston.

6. A syringe device according to claim 4, wherein the first and the second couplings are dimensioned such that when the injector piston is moved in the proximal direction, the reservoir piston is moved in the distal direction such that the increase in volume of the injector chamber substantially corresponds to the decrease in volume of the reservoir.

7. A syringe device according to claim 2, wherein the motor is coupled to the spring via a gear mechanism.

8. A syringe device according to claim 2, wherein the spring is arranged to be strained rotationally so as to store energy.

9. A syringe device according to claim 2, wherein the spring is arranged to be strained translationally so as to store energy.

10. A syringe device according to claim 2, further comprising a release mechanism adapted to be changed between a retaining position wherein it retains the spring in its strained state, and a releasing position wherein the spring is allowed to be unstrained.

11. Use of a motor to strain a spring of a syringe device according to claim 2.
